(19) United States
(12) Patent Application Publication
(10) Pub. No.: US 2003/0208189 A1
(43) Pub. Date: Nov. 6, 2003

(54) Integrated system for correction of vision of the human eye

(76) Inventor: Gholam A. Payman, New Orleans, LA (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/193,266

(22) Filed: Jul. 12, 2002

Related U.S. Application Data
(63) Continuation-in-part of application No. 09/982,079, filed on Oct. 19, 2001.

(51) Int. Cl. A61B 18/20

(52) U.S. Cl. 606/5

(57) ABSTRACT

A system and method for correcting vision in the eye, including an imaging device adapted to form images of a portion of the eye and transmit the images to a remote location. A display device is in communication with the imaging device and is adapted to display the images of the portion of the eye at the remote location. A laser is adapted to be positioned relative to the eye and coagulate the portion of the eye displayed on the display device. A control device is at the remote location and is adapted to control the position of the laser.

### FIELD OF THE INVENTION

The present invention relates to a system and method for correcting the vision of the eye. More particularly, the present invention relates to a system and method for correcting the vision in the eye using robotic arms and remote control to assist in correcting the vision in the cornea and/or in the retina.

### BACKGROUND OF THE INVENTION

A conventional method for correcting the refractive error in a cornea is keratophakia, i.e., implantation of a lens inside the cornea. Keratophakia uses an implant, which is placed into the cornea approximately equidistant from the exterior surface of the cornea and the interior surface. The procedure is usually done by first preparing a lens from corneal donor tissue or synthetic material using a cryo-lathe. The lens is implanted by removing a portion of the cornea with a device called a microkeratome, and the tissue is sutured back into place over the lens. However, there can be problems when microkeratomies are used for cutting the cornea. First, irregular keratectomies or perforations of the eye can result. Second, the recovery of vision can be rather prolonged.

Another surgical technique exists that uses a femtosecond laser to separate layers inside the stroma, at least two-thirds of the distance from the top surface of the cornea to the inside of the eye. An incision is made to access this area and a solid inlay is inserted to help correct myopia in the eye. However, by separating the layers in the bottom two-thirds of the stroma, it is difficult to access the separated area to insert the inlay and virtually impossible to change or modify the inlay without another extensive surgical procedure. This procedure also requires making an incision, which is parallel to the visual axis and is limited in the lateral direction by a maximum size of 0.3 mm, to encase a relatively rigid inlay that forces the tissue in the lateral direction.

Additional surgical techniques exist that use ultraviolet light and short wavelength lasers to modify the shape of the cornea. For example, excimer lasers, such as those described in U.S. Pat. No. 4,840,175 to Peyman, which emit pulsed ultraviolet radiation, can be used to decompose or photoablate tissue in the live cornea so as to reshape the cornea. The entire content of U.S. Pat. No. 4,840,175 is incorporated by reference herein.

Specifically, the Peyman patent discloses the laser surgical technique known as laser in situ keratomycosis (LASIK). In this technique, a portion of the front of the live cornea can be cut away in the form of a flap having a thickness of about 160 microns. This cut portion is removed from the live cornea to expose an inner surface of the cornea. A laser beam is then directed onto the exposed inner surface to ablate a desired amount of the inner surface up to 150-180 microns deep. The cut portion is reattached over the ablated portion of the cornea and assumes a shape conforming to that of the ablated portion. Additionally, in the Lasik procedure, a femtosecond laser can be used to cut and separate the flap.

However, because only a certain amount of cornea can be ablated without the remaining cornea becoming unstable or experiencing outbulging (eklasisa), this technique may not be especially effective in correcting very high myopia. That is, a typical cornea is on average about 500 microns thick. The laser ablation technique generally requires that at least about 250 microns of the corneal stroma remain after the ablation is completed so that instability and outbulging do not occur.

Additional methods for correcting the refractive error in the eye include inserting an implant between layers of the cornea. Generally, this is achieved using several different methods. One method involves inserting a ring between layers of the cornea, as described in U.S. Pat. No. 5,405,384 to Silvestrini. Typically, a dissector is inserted in the cornea and forms a channel therein. Once it is removed, a ring is then inserted into the channel to alter the curvature of the cornea. In another method, a flap can be created in a manner similar to the LASIK procedure, and a lens can be inserted under the flap, as described in U.S. Pat. No. 6,102,946 to Nigam. A further method involves forming a pocket using an instrument, and inserting an implant into the pocket, as described in U.S. Pat. No. 4,655,774 to Choyce. The entire contents of U.S. Pat. Nos. 4,655,774, 5,405,384 and 6,102,946 are incorporated by reference herein.

However, with the above-described techniques, a knife or other mechanical instrument is generally used to form the channel, flap or pocket. Use of these instruments may result in damage or imprecision in the cut or formation of the desired area in which the implant is placed.

Furthermore, many of the steps of the above procedures are performed by hand. For example, the formation of the flap and the placing of the intraocular lens on the surface of the cornea are generally performed by the surgeon performing the operation. By performing these tasks by hand, there can be a relatively large margin of error in which the surgeon may improperly form the flap or improperly position the lens.

Additionally, many conventional excimer lasers are permanently or semi-permanently fixed, and the patient is positioned in front of the laser during the procedure. This restricts the work environment and can result in difficulties in obtaining the proper positioning of the patient.

Other methods for correcting the vision in the eye include ophthalmic laser surgery at the back or retina of the eye. This type of procedure is generally performed on a slit-lamp equipped with a laser. The surgeon uses a contact lens to visualize the fundus or eye background and a joystick to move a laser over the desired portion of the retina. Once in the proper position, the surgeon activates the laser to coagulate the desired portion of the retina.

However, as with the procedures described above, the physician must be physically present at the treatment site to perform the procedure. Thus, patients living in remote areas of the world may not have the benefit of a surgeon, let alone an experienced, qualified surgeon.

Therefore, there exists a need for an improved, more versatile method of correcting vision in the eye.

### SUMMARY OF THE INVENTION

Accordingly, it is an object of the present invention to provide an improved, more versatile method of correcting vision in the eye.

Another object of the present invention is to provide a system and method that allows remote modification of the eye.

Still another object of the present invention is to provide a system and method for remote modification of the retina.

Yet another object of the present invention is to provide a system and method for remote modification of the cornea.

The foregoing objects are basically attained by a system for correcting vision in the eye, including an imaging device adapted to form images of a portion of the eye and transmit said images to a remote location. A display device is adapted to display the images of the portion of the eye at the remote location and a laser is adapted to be positioned relative to the portion of the eye and coagulate the portion of the eye. A control device is at the remote location and is adapted to control the position of the laser.

The foregoing objects are further attained by a method for remote correction of vision in the eye. The method includes the steps of forming an image of the retina of the eye and aiming a laser at the retina of the eye. The image of the retina is transmitted to a remote location and displayed at the remote location. Information is transmitted from the remote location to the laser to move the laser relative to the eye; and then information is transmitted from the remote location to the laser to fire the laser and coagulate a portion of the retina.

The foregoing objects are further attained by a system for correcting vision in the eye, including an imaging device adapted to take images of a portion of the eye and a laser adapted to be positioned relative to the eye and be fired at said portion of the eye. A first transmitting device is coupled to the imaging device and the laser, and the first transmitting device is adapted to transmit the images of the portion and the position of the laser to a remote location. A first receiving device is at the remote location and is adapted to receive the information transmitted by the first transmitting device. A first monitor is also at the remote location and is coupled to the first receiving device for displaying the images of the portion. A first control device is at the remote location and is adapted to control the position of the laser. A second transmitting device is at the remote location is coupled to the control device and is adapted to transmit control signals from the first control device. A second receiving device is coupled to the laser and is adapted to send the control signals to the laser to move the laser.

Other objects, advantages, and salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 illustrates a system for correcting refractive error in the cornea of the eye, according to an embodiment of the present invention;

FIG. 2 is a top perspective view of a robotic arm with a excimer laser coupled thereto;

FIG. 3 is a top perspective view of a robotic arm with a ultrashort pulse laser coupled thereto;

FIG. 4 is a bottom perspective view of a robotic arm with the lens dispensing device of FIG. 9 coupled thereto;

FIG. 5 is a side cross-sectional view of the eye of a patient with a corneal flap formed in the cornea of the eye by irradiating the cornea with an ultrashort pulse laser;

FIG. 6 is front elevational view of an internal corneal surface of the cornea with a mark in the shape of a dot aligned with the optical axis;

FIG. 7 is front elevational view of an internal corneal surface of the cornea with a mark in the shape of a “plus” aligned with the optical axis;

FIG. 8 is a side cross-sectional view of an intracorneal lens positioned on an exposed internal corneal surface of the eye of FIG. 5;

FIG. 9 is a side cross-sectional view of a lens dispensing device with intracorneal lenses inserted therein;

FIG. 10 is front elevational view of an intracorneal lens with a mark in the shape of a dot aligned with the central axis of the lens;

FIG. 11 is front elevational view of an intracorneal lens with a mark in the shape of a “plus” aligned with the central axis of the lens;

FIG. 12 is a side cross-sectional view of a laser ablating a portion of the intracorneal lens of FIG. 8;

FIG. 13 is front elevational view of a ring-shaped intracorneal lens with a portion having a cross-section area larger than another portion;

FIG. 14 is a side cross-sectional view of the lens of FIG. 13, taken along line 14-14;

FIG. 15 is front elevational view of a ring-shaped intracorneal lens with a portion of the ring ablated or removed;

FIG. 16 is a side cross-sectional view of the lens of FIG. 15, taken along line 16-16;

FIG. 17 is a side cross-sectional view of a second embodiment of the present invention, wherein a portion of the intracorneal lens that is offset from the optical axis is ablated by a laser;

FIG. 18 is a side cross-sectional view of a third embodiment of the present invention, wherein the outer periphery of the intracorneal lens is ablated by a laser;

FIG. 19 is a side cross-sectional view of the corneal flap repositioned over the intracorneal lens of FIG. 16;

FIG. 20 is a side cross-sectional view of the corneal flap repositioned over the intracorneal lens of FIG. 17

FIG. 21 is a side cross-sectional view of the corneal flap repositioned over the intracorneal lens of FIG. 18;

FIG. 22 is a side cross-sectional view of a compression device compressing the repositioned corneal flap of FIG. 19, to smooth any wrinkles or folds in the flap;

FIG. 23 is a side cross-sectional view of a therapeutic contact covering the repositioned corneal flap of FIG. 22;

FIG. 24 is front elevational view of the external surface of the cornea with a mark in the shape of a “plus” aligned with the optical axis;

FIG. 25 is front elevational view of the external surface of the cornea with a mark in the shape of a dot aligned with the optical axis;

FIG. 26 is a schematic of a system for remote correction of vision in the eye using a wireless communication system; and

FIG. 27 is a schematic of a system for remote correction of vision in the eye using a wired communication system.

### DETAILED DESCRIPTION OF THE INVENTION

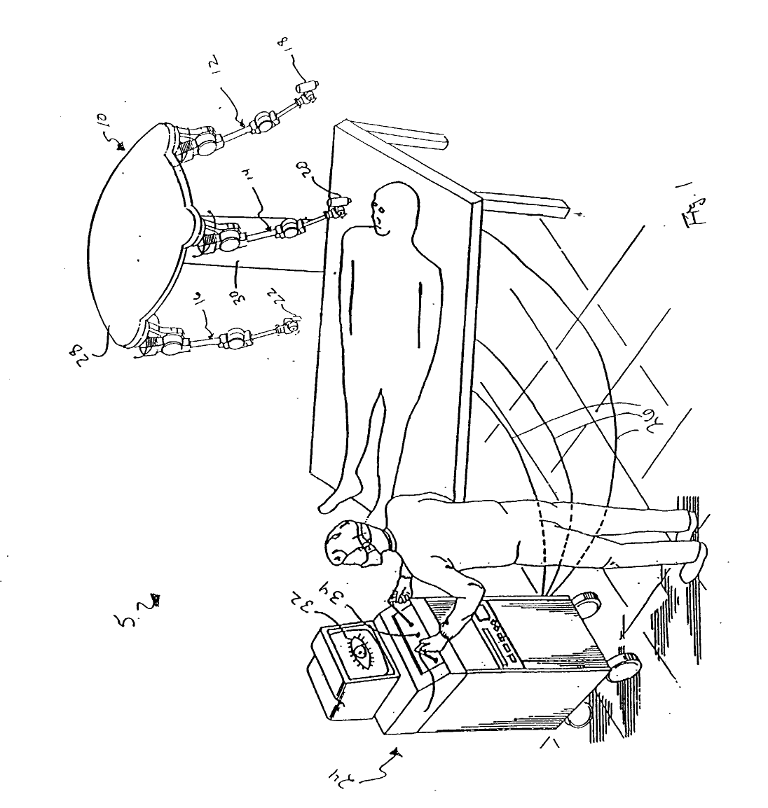

FIG. 1 illustrates an example of an integrated system for correcting the refractive error of an eye according an embodiment of the present invention. The system 5 includes device 10 that has at least a first robotic arm or automated device 12, a second robotic arm or automated device 14 and a third robotic arm or automated device 16 coupled thereto. First robotic arm 12 has an ultrashort pulse laser 18 coupled thereto, second robotic arm 14 has an excimer laser 20 coupled thereto and third robotic arm 16 has a lens dispenser 22 coupled thereto. Each device and each robotic arm can be controlled remotely at station 24.

Device 10 preferably is electrically connected to station 24 using electrical cables 26 or any other manner known in the art. Furthermore, device 10 has a top portion 28 and a support structure 30. Device 10 is capable of moving in any direction desired using wheels (not shown) or any other suitable moving means, such as a track that would allow it to move as desired. Top portion 28 can rotate relative to structure 30, thus allowing device 10 to position itself or the individual robotic arms in any of a variety of ways.

Station 24 preferably is a movable computer control device that has a monitor 32 and controls 34. Controls 34 allow the surgeon to move and position the device 10 and each individual robotic arm to the proper location for use in the desired surgical technique. Monitor 32 provides the surgeon with a detailed view of the area, which is the subject of the specific surgical step, to achieve this display, each robotic arm preferably has a small camera (not shown), which allows the monitor 32 of the station 24 to display project the specific area that is the subject of the specific surgical step.

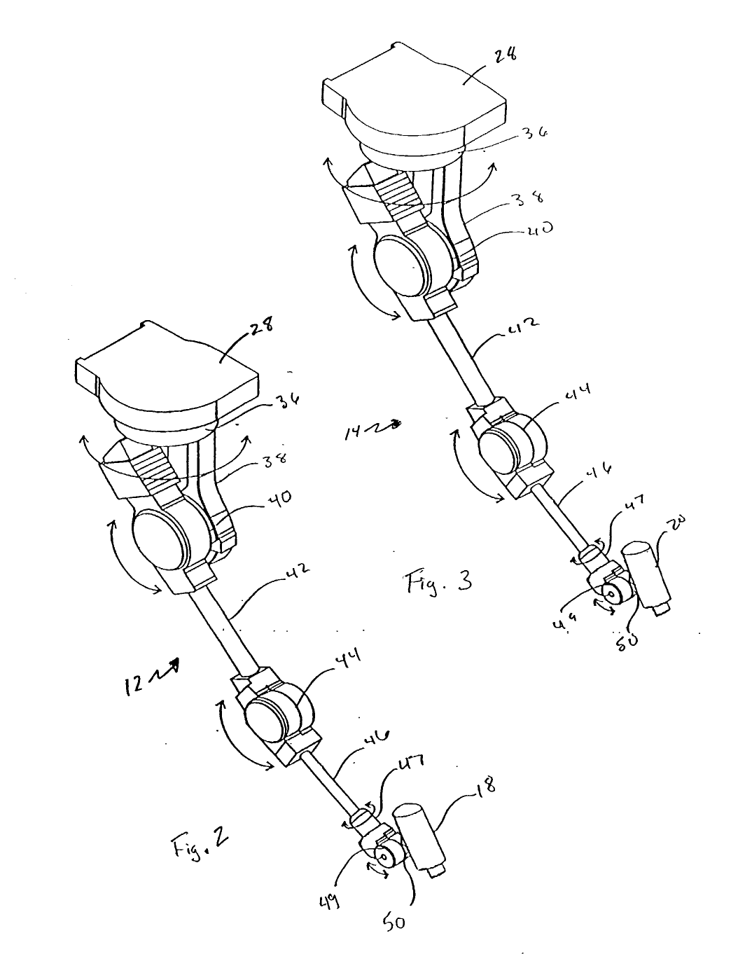

As shown in FIGS. 2, 3 and 4, each robotic arm 12, 14 and 16 is substantially similar and therefore, only arm 12 will be described herein. Arm 12 is coupled to top portion 28 by a rotatable joint 36, which allows upper portion 38 of arm 12 rotate 360 degrees relative to top portion 28. Additionally, arm 12 has a shoulder joint 40, which connects the upper portion 38 to arm portion 42 and an elbow joint 44, which connects arm portion 42 with lower arm portion 46. Both joints 40 and 44 can move about at least 180 degrees or more, as desired. Furthermore, arm 12 has a first wrist joint 46 and a second wrist joint 47. First wrist joint 47 allows end portion 50 to rotate 360 degrees and second wrist joint 49 allows the end portion 50 to move about 180 degrees or more, as desired, relative to lower arm portion 46. Laser 18 is coupled to end portion 50 in any conventional manner as desired. By having a variety of joints and arm portions as described above, the arm 12 can move in a similar fashion to a human arm and is not limited to a specific area in which a patient must be positioned to perform the apparatus which will now be described.

To begin, the refractive error in the eye is measured using wavefront technology, as is known to one of ordinary skill in the art. A more complete description of wavefront technology is set forth in U.S. Pat. No. 6,086,204 to Magnate, the entire contents of which is incorporated herein by reference. The refractive error measurements are transmitted to a computerized lathe (not shown) or other lens-shaping machine, where the shape of ocular material used to form the blank or corneal implant is determined based on the information from the wavefront device. Alternatively, the ocular material 52 can be manufactured or shaped prior to the use of the wavefront technology and can be stored in a sterilized manner until that specific shape or size is needed.

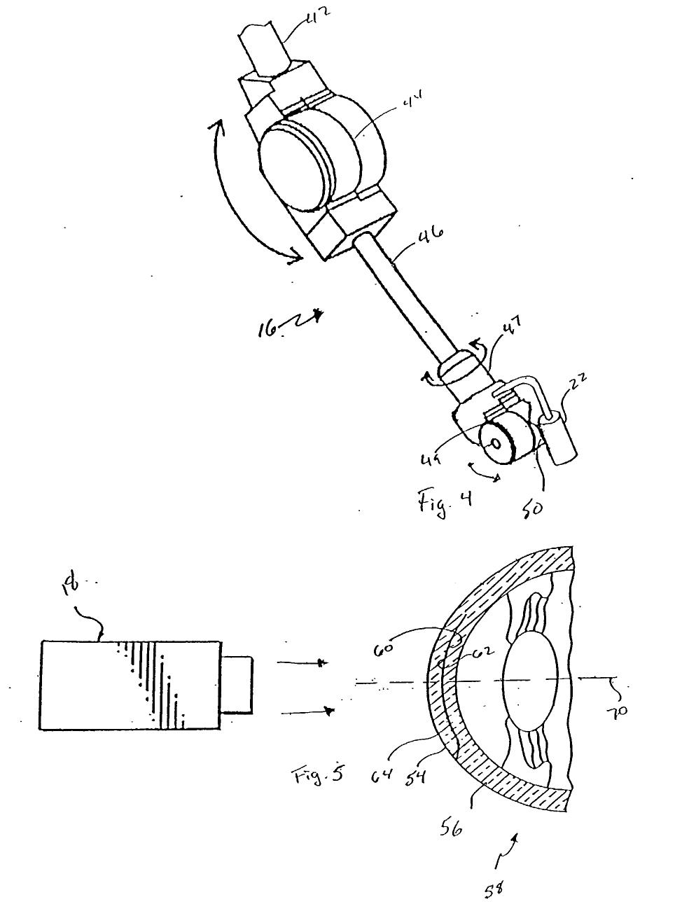

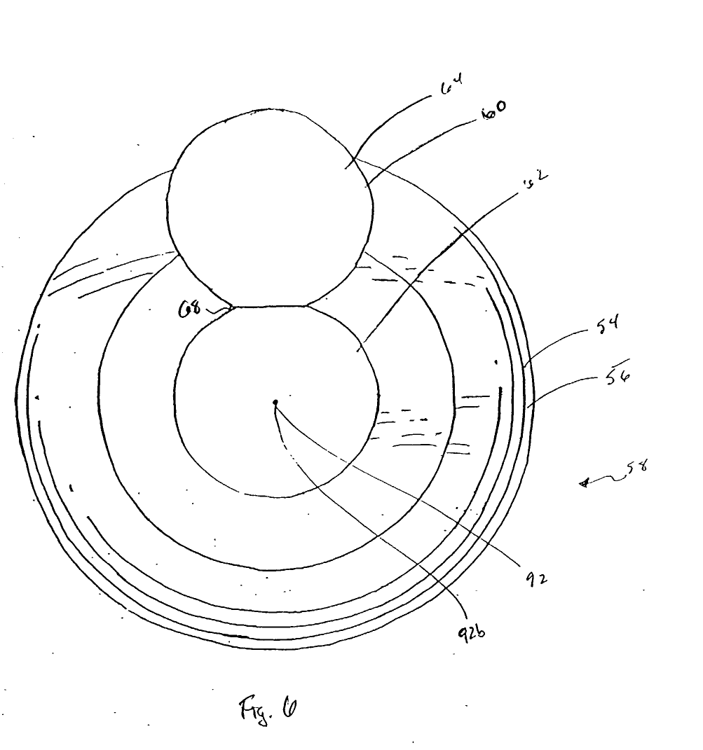

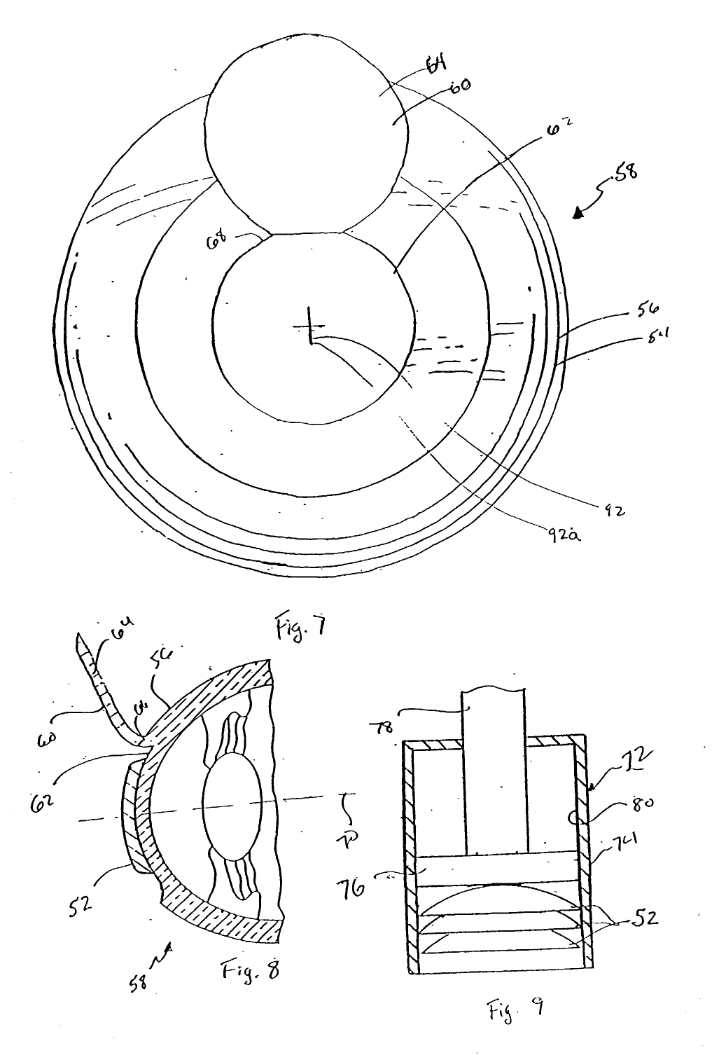

As shown specifically in FIG. 5, a laser 18 is aimed at the surface 54 of the cornea 56 of the eye 58 and energized under the control of station 24 and robotic arm 12. The laser preferably separates the internal area of the cornea into first internal surface 60 and second internal surface 62, which are both substantially circular and form the substantially circular corneal flap 64. First internal surface 60 faces in a posterior direction of cornea 56 and the second internal surface 62 faces in an anterior direction of the cornea 56. The flap 64 preferably has a uniform thickness of about 10-250 microns, and more preferably about 80-100 microns, but can have any suitable thickness. A portion 66 of flap 64 preferably remains attached to the cornea by an area located at the perimeter 68 of flap 64, as shown in FIGS. 6 and 7. However, the laser 18 can form a flap of any suitable configuration, such a flap attached at portion surrounding the main optical axis 70 or any other suitable location or a flap that is not attached to the cornea at all. Additionally, the flap 64 may be shaped or sized as desired and does not need to be circular.

Laser 18 is preferably an ultrashort pulse laser, such as a pico, femto or atosecond laser, but may be any light emitting device suitable for creating a flap in the cornea 56. The ultrashort pulse laser is positioned in front of the eye 58, using the control station 24 and robotic arm 12, and focuses the laser beam in the cornea 56 at the desired depth and in the desired flap configuration. Ultrashort pulse lasers are desired since they are high precision lasers that require less energy than conventional lasers to cut tissue and do not create “shock waves” that can damage surrounding structures. Cuts made by ultrashort pulse lasers can have very high surface quality with accuracy better than 10 microns, resulting in more precise cuts than those made using mechanical devices or other types of lasers. This type of accuracy results in less risks and complications than the procedures using other types of lasers or mechanical devices.

As seen in FIG. 8, the flap 64 is then lifted using any device known in the art, such as spatula or microforceps (not shown) or any other device. The microforceps can be coupled or attached to a robotic arm, as described above, which would allow the flap to be lifted under the control of station 24, or the microforceps can be manually manipulated by the surgeon performing the surgical procedure.

Lens or implant blank 52 is then positioned or introduced in between the first and second internal surfaces 60 and 62, respectively of the flap 64. Preferably, the implant is positioned or placed on internal corneal surface 62 using implant dispenser or carrier device 22, as shown in FIG. 9. Specifically, the lens dispenser 22 has a housing 74, a plunging portion 76 and a stem 78. Preferably the entire device 72 is formed of transparent plastic and the housing has an outer diameter of about 1-11 mm. The plunging portion is also transparent plastic with a diameter of about 1-9 mm, which is substantially the same as both the inner diameter 80 of housing 74 and the diameter of implant 52. The plunging portion 76 also can have a mark, such as “cross hairs” or a dot centered thereon, which helps in aligning the implant 52 with the center or optical axis of the eye 58. Since the housing 74, stem 78 and plunging portion 76 can each be transparent, by putting “cross hairs” or a dot on the plunging portion a camera or the naked eye can be used to view the “cross hairs” through the housing and/or stem and the plunging position to align the contact with the surface of the eye. The stem 78 electrically connects the plunging portion to station 24 so that station 24 can control when an implant is placed on the surface of the cornea. The plunger is generally coupled to an electric or pneumatic (or any other type of motor) which can be activated remotely to actuate the plunger mechanism and thereby deposit an implant on the surface of the eye. However, the plunging mechanism does not necessarily need to be motorized and can be manually activated by hand if desired. The carrier device 72 can carry at least one implant 52 therein and preferably carries more than one implant therein, such as three or more. Thus, each eye of a patient may be operated on in succession without reloading the implant dispenser 22 or multiple patients may be operated on in succession without reloading the dispenser.

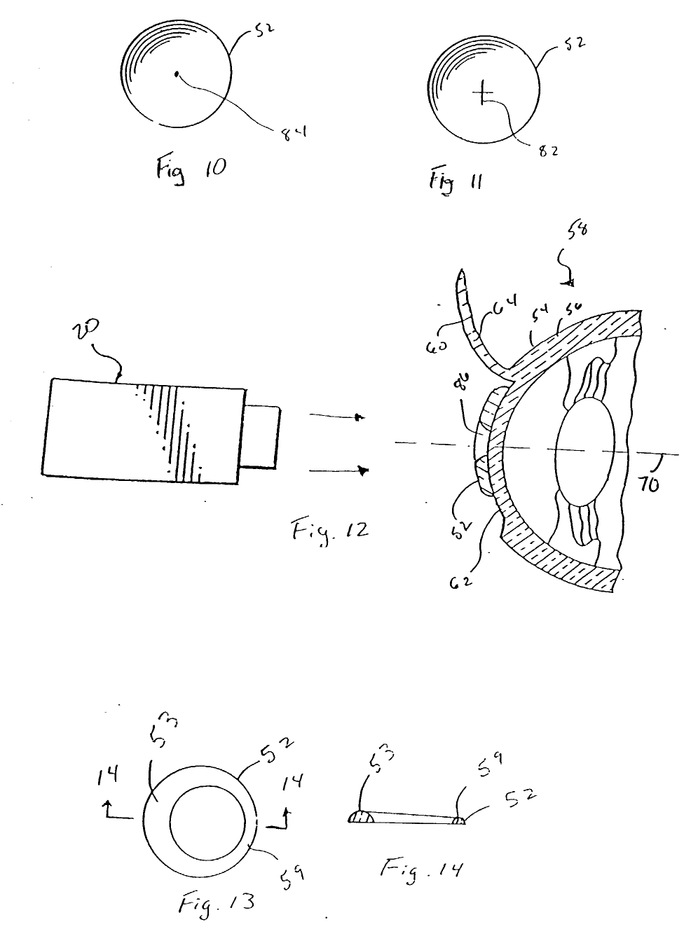

Implant 52 is preferably any polymer or hydrogel having about 50% water content; however, the water content can be any percentage desired. The implant may be formed from synthetic or organic material or a combination thereof. For example, the implant can be collagen combined with or without cells; a mixture of synthetic material and corneal stromal cells; silicone or silicone mixed with collagen; methylmetacrylate; any transparent material, such as polyprolidine, polyvinylpylidine, polyethylenoxyde, etc.; or any deformable polymer, which can change its shape with radiation after implantation. Additionally, the implant can be any shape or sized desired, can have a different or similar refractive properties to the refractive properties of the cornea, and it can have pigmentation added thereto to change the color of the lens or it can be photochromatic. As seen in FIGS. 10 and 11, the implant 52 can have a mark, such as “cross hairs” 82 or a dot 84 centered thereon, which helps in aligning the lens with the center of the eye.

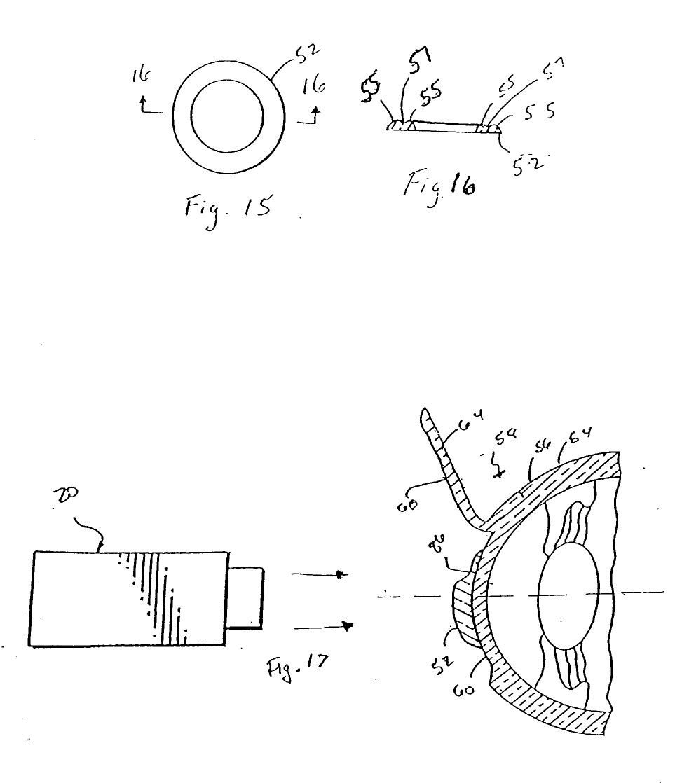

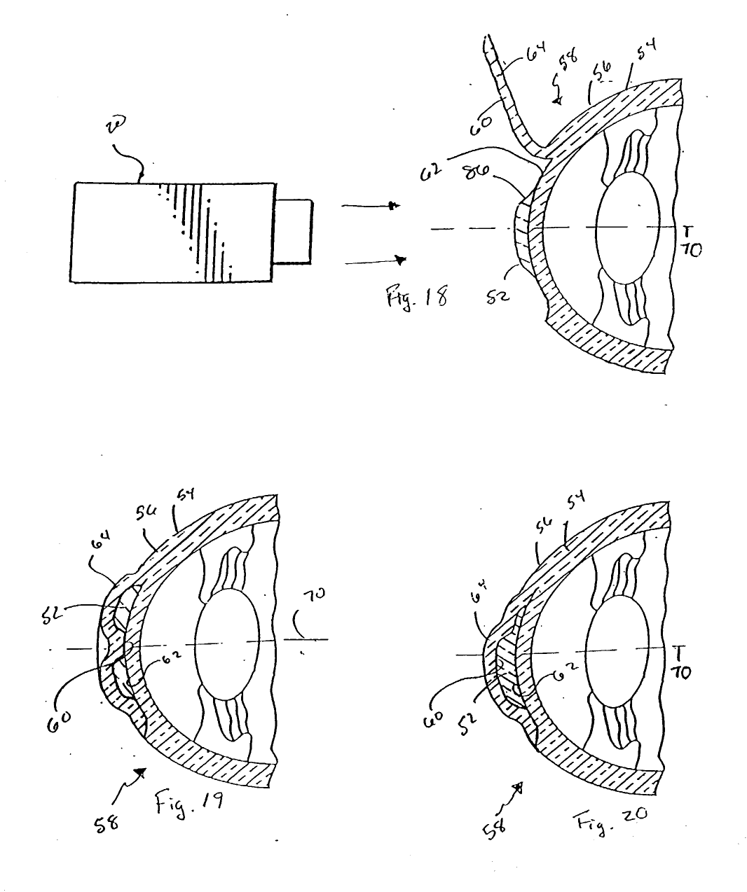

Additionally, as seen in FIGS. 12-14 once the implant 52 is in place, if necessary, the implant can be irradiated and a portion 86 of the ocular material can be ablated by an excimer laser 20. The implant 52 can be ablated to form any shape or size desired. Specifically, the implant 52 preferably is about generally about 1-9 mm about 1-9 mm in diameter when placed on the surface of the cornea. The curvature preferably has a radius of about 1-20 mm, and more preferably about 4-7 mm. The thickness is preferably about 5-2000 microns, and more preferably about 100 microns. As seen in FIG. 12 and after ablation, the lens 52 is about 1-9 mm from the inner diameter to the outer diameter and more preferably about 3-5 mm. Additionally, the inside edge can be ablated to be thinner or thicker than the outside edge; for example the inside edge can have a thickness of about 1-100 microns, while the outside edge has a thickness of about 20-3000 microns. However, the implant can have any thickness or configuration that would allow it to elevate or move any portion of the flap 64 relative to surface 62. For example, the lens 52 can be ablated to form a ring having multiple bumps or “peaks 55 and valleys 57” (FIGS. 13 and 14) or the lens can be partially ablated, so that one portion 53 of the ring has a larger cross-sectional area than another section 59 (FIGS. 15 and 16).

Furthermore, the lens 52 can have an area at the outer perimeter ablated (FIG. 18) or it can have one side or portion offset from the main optical axis ablated (FIG. 17), to help correct astigmatism. The thickness ablated, amount ablated and position of lens 52 generally defines the degree of correction.

It is noted that although it is preferable to have the inlay ablated on or adjacent to the surface of the cornea, the inlay can be formed or shaped in any manner desired. For example, the inlay can be formed or shaped away from the cornea using a laser, a mill or casting. Preferably, these shaping methods are done when the information needed to modify the inlay or lens is transmitted through a computerized system after wavefront technology has measured the refractive error of the eye; however, as stated above, the wavefront technology information may be used to determine the specific shape and/or size of the prefabricated lens that is to be used.

As noted above, laser 20 can be an excimer laser. Excimer lasers are generally known in the art for being capable of ablating both corneal tissue and synthetic material. However, since excimer lasers are generally developed for ablation of the cornea, there are expensive to produce, require toxic fluorine gases, and are difficult to maintain. Therefore, it may be preferable to ablate the implant 52 using lasers that are cheaper and easier to maintain. Certain lasers that produce a wavelength of about 355 nm can be cheaper and easier to maintain. However, it is noted that the laser can emit a beam having a wavelength of about 355 nm to about 1300 nm.

Preferably, when using this type of laser, the implant is ablated, producing holes in the polymer, without producing a coagulative effect on the material. The 355 nm photon has three times the energy of the conventional 1064 nm photon, enabling the 355 photon to break molecular bonds. The 355 nm wavelength can be generated using a diode pumped solid-state (DPSS) Nd-YAG laser, which is double frequencied to 532 nm and mixed with a Nd-YAG at 1064 nm, producing the 355 nm wavelength.

Additionally, the combination of a diffraction-linked beam and a short wavelength laser can enable machining of the implant, since the focal spot size is proportional to the wavelength. For example, the laser can emit a short pulse or ultrashort pulse of a picosecond, a nanosecond, a femtosecond or an attosecond. However, the laser can be any suitable continuous or pulsed laser, or any laser that emits a beam in the infrared or visual spectrum.

Preferably, when utilizing this type of frequency laser, a flying spot laser is used, which can be moved though a software program across the inlay to ablate the desired portion of the implant.

Furthermore, it is noted that this type of procedure and this type of 355 nm wavelength laser can be used to alter or ablate any type of lens, implant or inlay known in the art. For example, suitable eye correcting devices are intracorneal inlays, intraocular implants, contacts positioned on the surface of the cornea, and any other suitable device.

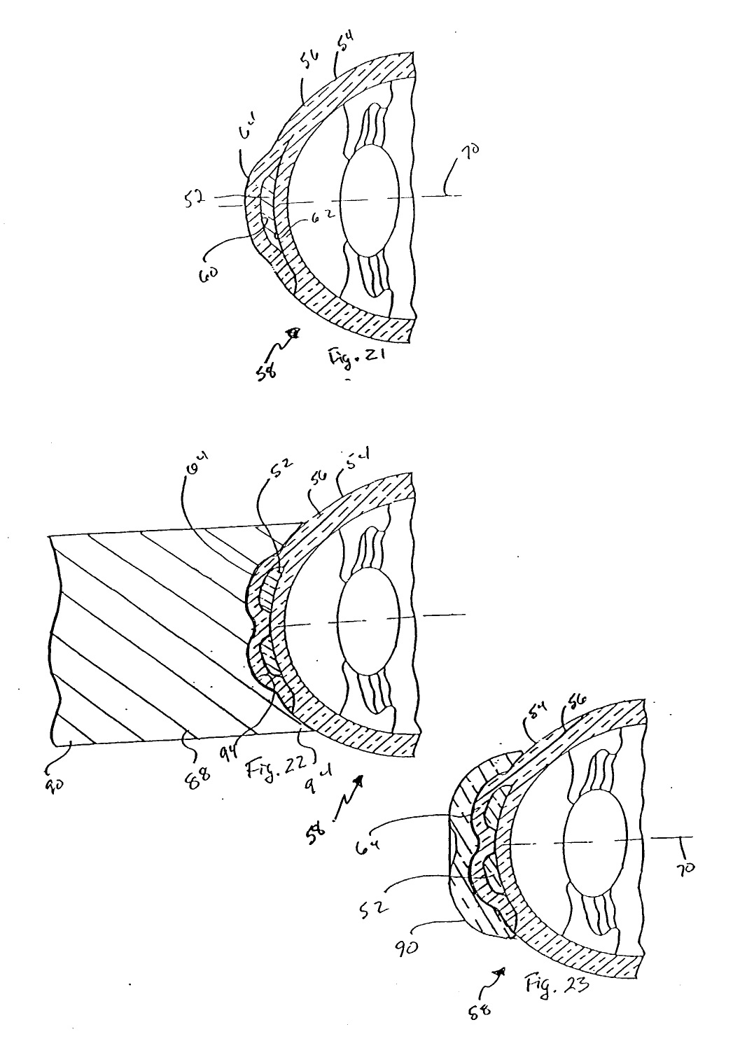

Once the implant has been altered or ablated to the desired shape, the flap 64 is replaced so that it covers or lies over the lens in a relaxed state, as seen in FIGS. 19-21. In other words, implant 52 does not force flap 64 away from the internal surface 62, and described above, and therefore the refractive properties of the cornea are not altered due to a tension force being applied to the flap.

After ablation and repositioning of the flap 64, a compression device or polymeric cylinder 88 can be applied to the external surface of the cornea, as seen in FIG. 22. The polymeric cylinder can be formed from any polymer and is preferably silicon, or a silicon sponge or any combination of silicon and other polymers. Cylinder 88 has a first end 90 and a second end 92. The end surface 94 adjacent second end 92 preferably has the shape that is substantially similar to the outer surface shape of lens 52. The cylinder 88 smoothes the inlay and the corneal flap by having gentle pressure applied thereto. Preferably, the pressure is slightly greater than the normal pressure required for suctioning the cornea top create a flap using a microkeratome as is known to one skilled in the art. However, the pressure can be any desired pressure that would effectively smoothen the flap, including normal intraocular eye pressure.

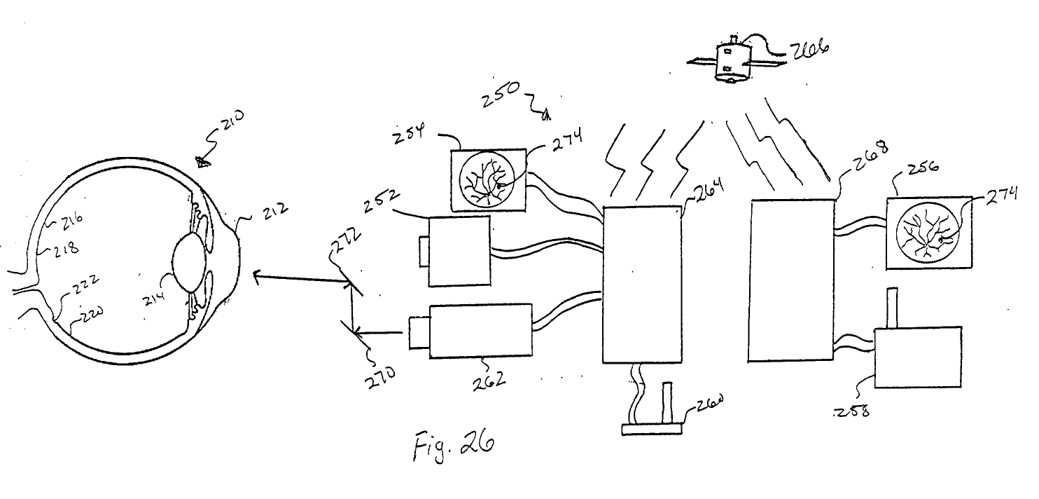

At the end of the procedure or before the ablation of the surface of the cornea, topical agents, such as an anti-inflammatory, antibiotics and/or an antiprolifrative agent, such as mitomycin or thiotepa, at very low concentrations can be used over the ablated area to prevent subsequent haze formation. The mitomycin concentration is preferably about 0.005-0.05% and more preferably about 0.02%. A short-term bandage contact lens 90 may also be used to protect the cornea, as seen in FIG. 23.

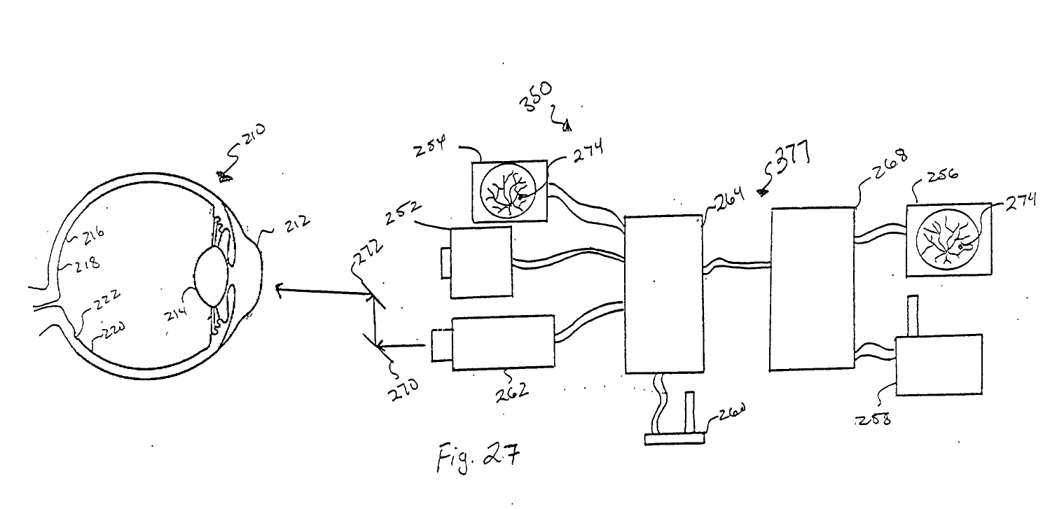

Furthermore, as seen in FIGS. 24 and 25, a mark 92 such as “cross hairs” 92a (FIG. 24) or a dot 92b (FIG. 25) can be placed on the eye. This can be done using a laser or physically applying a mark 4 using a dye or any other desired substance. This mark can help in aligning the position in which a laser is to form the flap. Additionally, as seen in FIGS. 6 and 7, a similar mark 92 may be made on surface 62, it is easier to align the mark on lens 52 with the area on the surface of the cornea where lens 52 will be placed. Furthermore, when using station 24 and robotic arm 16, the mark on the lens and/or on the lens and the dispensing device 22 can be aligned with mark 92 which would ensure an accurate placement of lens 52.

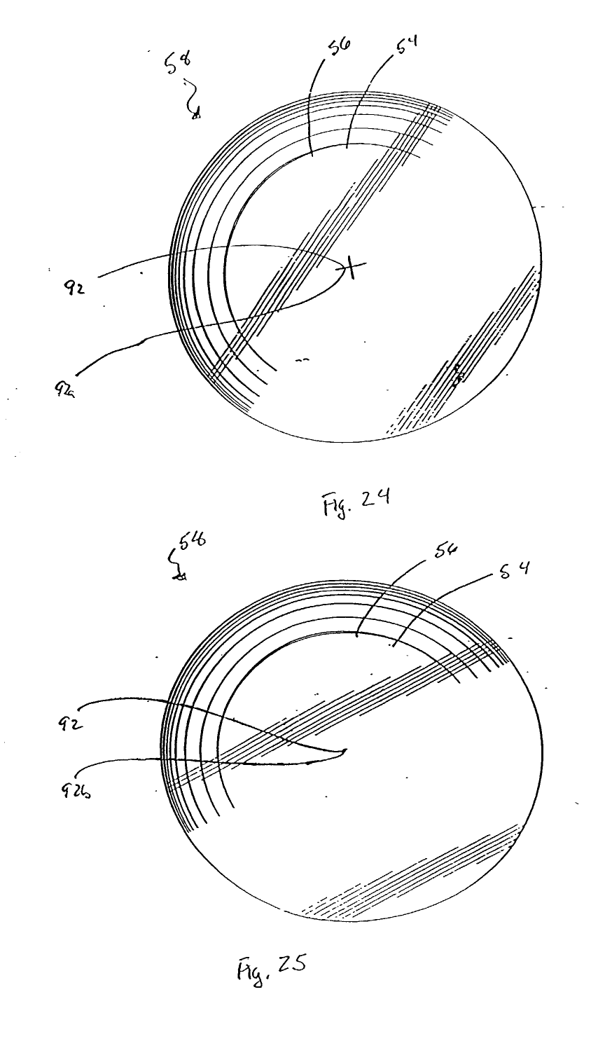

As more fully described in the above referenced applications, U.S. application Ser. No. 09/758,263 and U.S. application Ser. No. 09/797,177, the exposed surface of the cornea, preferably the stroma can be ablated to further correct the refractive error in the eye for any condition such myopia, hyperopia or astigmatism. For example, the refractive error in the eye is measured using wavefront technology. A flap 18 is then formed in the surface of the cornea and a portion of the exposed surface is ablated, either concentric with the main optical axis or offset from the main optical axis can be ablated to correct the specific problem. As shown in FIG. 21, an implant or lens 120 can then be positioned as described above. Preferably implant 120 has predetermined refractive properties for myopic or hyperopic or astigmatic correction. Implant 120 can be substantially similar to implant 20 or implant 56 or any other desired shape. Furthermore, as seen in FIG. 22, lens 120 can be ablated in any manner desired to further correct myopic, hyperopic or astigmatic error in the eye, as described above.

Flap 18 can then be repositioned over the exposed surface and the implant 120 in a relaxed state, as shown in FIG. 23, and similar to the repositioning of the flap described above.

It is noted that lasers 18 and 20 and lens dispensing device 22 do not necessarily need to be on separate robotic arms and may be all on the same arm or any two devices on the same arm. In this configuration, a rotating device could be placed at the end of the robotic arm that would allow the surgeon to position the proper device in front of the patient's cornea and activate each device accordingly.

When correcting either the implant or ablating a portion of the stroma with the excimer laser, it is possible to simultaneously use wavefront technology or Adaptec optic technology to create a near perfect correction in the eye and to remove all corneal irregularities. By using this technique to correct vision, it is possible to achieve 20/10 vision in the patient's eye or better.

Embodiment of FIGS. 26 and 27

FIGS. 26 and 27 illustrate a system and method for remote correction of vision in the eye. As shown specifically in FIG. 26, the eye 210 has a cornea 212, a lens 214 and a fundus 216. The findus includes the retina 218, the macula 220, the fovea 222, and other components of the eye such as the optic disc and retinal vessels.

The vision in the eye 210 can be corrected remotely using a system 250 that preferably forms an image of the fundus 216 using a camera or imaging device 252. The image is displayed both at the location of the procedure and at a remote location. The image at the location of the procedure is displayed using a monitor or a second display device 254, and is also transmitted to a second monitor or first display device 256 at the remote location. Once the image is displayed on either or both of the monitors 254 and 256, an operator can determine the area of the fundus 216 that needs to be corrected, and using a first control device 258 at a remote location or a second control device 260 at the location of the procedure, manipulate a laser 262 to coagulate a portion of the fundus or more preferably, the retina.

More specifically, the camera 252 preferably takes an image of the fundus of the eye and transmits the information to a device 264 that can store the information. Device 264 can then transmit the information to monitor 254 and/or transmit the information, via satellite 266, to a device 268 that is capable of receiving information or data at the remote location. The device 268 then transmits the image to monitor 256, which in turn displays the image of the fundus.

Laser 262 is preferably a laser as described above for laser 20, and any description of laser 20 is applicable to laser 262. Furthermore, laser 262 can be a coagulative laser, a disruptive laser, an ablative laser, a thermic laser (without coagulation) or a laser that is used to activate photo sensitizers in the body of a patient. Laser 262 is, along with camera 252, aimed at the fundus of the eye. Laser 262 can be positioned to project directly into the eye or it can use a series of mirrors or reflecting devices, such as mirrors or devices 270 and 272, that would allow the laser to be positioned as to not hinder the view of the camera 252, or in any position desired. Preferably, the laser projects an indicator 274 onto the fundus of the eye, so that the exact location in which the laser light will strike the fundus of the eye is indicated on the monitors 254 and 256. The indicator can be any type desired, such as visible light, infrared light or any light that can be converted and displayed visually on a monitor.

Using the indicator 274 as a guide, the operator at the remote location or at the sight of the procedure can use a control mechanism or device 258 or 260, respectively, to control the laser position. Control devices 258 and 260 are preferably joysticks that allow movement of the laser and indicator to any position on the fundus and/or retina. However, it is noted that control device 258 and 260 do not necessarily need to be joysticks and can be voice activated control mechanisms. In other words, the position of the laser and/or the visual indicator can be changed or altered using a voice recognition system that moves the laser and/or the visual indicator in response to certain or predetermined voice commands, or the control device can be any other suitable control device.

Preferably, the control device 258 at the remote location is in communication, either by wireless or wired means, with device 268. Device 268 is not only capable of receiving information via satellite, but is also capable of storing information both received from device 264 or from the control device 258, and transmitting data, via satellite, to device 264.

Both devices 264 and 268 are similar and can receive data or information from an outside source, either via a wired connection or a wireless connection (i.e. satellite), store the information and transmit the information to a satellite. Applicant notes that it is not necessary to transmit/receive the information via satellite, and that the information can be transmitted/received using any wireless communication known in the art.

The information/data from the control device 258 is transmitted to the device 264, which in turn transmits the data to the laser 262. The laser then adjusts position to properly place the indicator in the position designated by the operator. It is noted that the control devices 258 and 260 can also control the position of the laser beam, so that the laser beam will contact the findus by changing the angle of the mirrors, and therefore the final position of the laser beam.

Additionally, the laser can be manipulated in the manner described above for FIGS. 1-25. In other words, the laser can change position in response to a control device moving a robotic arm to the desired position relative to the fundus of the eye.

Once the operator has properly located the indicator 274, using either the control device 258 or control device 260, the operator activates or fires the laser to coagulate a portion of the fundus, or more precisely, retina 218, as is known in the art.

It is noted that it is not necessary to connect the above-described devices in the specific order or manner described above. For example, each device does not need to be coupled to receiver 264 and/or 268. Each device can have its own receiver/transmitter or only a portion of the devices can be connected to the receiver. Furthermore, the receiver, the transmitter and the storage devices can be separate.

Additionally, it is not necessary to have a monitor at the location of the procedure.

As seen in FIG. 27, system 350 is substantially similar to system 250, except than the satellite 266 is replaced with a wired communication system 377. Wired communication system 377 can be any wired system desired, such as a cable system, the Internet or any other wired communication known in the art. All other reference numbers in the description of FIG. 26 are applicable to the FIG. 27.

Furthermore, the above-described system in FIGS. 26 and 27 can be implemented for corneal surgery as described above for FIGS. 1-25. In other words, the wireless or wired communication systems of FIGS. 26 and 27 can be used for the robotic arm system described above, or simply with the ablation of a lens or implant that is inserted into the cornea.

Additionally, it is noted that the procedure described herein is suitable surgical correction of other portions or structures in the eye, such as the lens capsule, the cornea, the anterior chamber angle or the iris, or any other suitable structure in the eye.

While preferred embodiments have been chosen to illustrate the invention, it will be understood by those skilled in the art that various changes and modifications can be made therein without departing from the scope of the invention as defined in the appended claims.

What is claimed is:

1. A system for correcting vision in the eye, comprising
an imaging device adapted to form images of a portion of the eye and transmit said images to a remote location;
a display device in communication with said imaging device and adapted to display said images of said portion of the eye at said remote location;
a laser adapted to be positioned relative to the eye and fired at said portion of the eye displayed on said display device; and
a control device at said remote location and adapted to control said position of said laser.

2. A system for correcting vision in the eye according to claim 1, wherein
said imaging device is a camera.

3. A system for correcting vision according to claim 1, wherein
said laser includes a visual marker, adapted to indicate the point in which a laser beam emitted from the laser would contact said portion of the eye.

4. A system for correcting vision according to claim 3, wherein
said display device is further adapted to display the visual marker indicating the point in which the laser beam emitted from the laser contacts said portion of the eye.

5. A system for correcting vision according to claim 1, wherein
said images are transmitted via wireless communication.

6. A system for correcting vision according to claim 5, wherein
said wireless communication is satellite communication.

7. A system for correcting vision according to claim 5, wherein
said control device transmits control information to said laser via wireless communication.

8. A system for correcting vision according to claim 7, wherein
said wireless communication is satellite communication.

9. A system for correcting vision according to claim 1, wherein
said images are transmitted via wired communication.

10. A system for correcting vision according to claim 9, wherein
said control device transmits control information to said laser via wired communication.

11. A system for correcting vision according to claim 1, wherein
said portion of the eye is the retina.

12. A system for correcting vision according to claim 1, wherein
said laser emits a laser beam in the infrared spectrum.

12. A system for correcting vision according to claim 1, wherein
said laser emits a laser beam in the visual spectrum.

13. A system for correcting vision according to claim 1, wherein
said laser is a continuous mode laser.

14. A system for correcting vision according to claim 1, wherein
said laser is an ultrashort pulse laser.

15. A system for correcting vision according to claim 14, wherein
said ultrashort pulse laser emits a pulse of at least one of the follow durations: nanosecond, picosecond, femtosecond or attosecond.

16. A system for correcting vision according to claim 1, wherein
said control system is a voice-activated system.

17. A method for remote correction of vision in the eye, comprising the steps of
forming an image of the retina of the eye;
aiming a laser at the retina of the eye;
transmitting the image of the retina to a remote location;
displaying the image of the retina at the remote location;
transmitting information from the remote location to the laser to move the laser relative to the eye; and
transmitting information from the remote location to the laser to fire the laser and coagulate a portion of the retina.

18. A method according to claim 17, wherein
each of the transmitting steps includes transmitting to a satellite.

19. A method according to claim 17, wherein
each of said transmitting steps includes transmitting via wired communication.

20. A method according to claim 17, wherein
each of said transmitting steps includes transmitting via wireless communication.

21. A method according to claims 17, wherein
the aiming the laser step includes aiming the laser so that a visual indicator appears on the retina of the eye at the point in which a laser beam emitted from the laser would contact the retina.

22. A method according to claim 21, wherein
the step of transmitting the image of the retina to a remote location includes transmitting the visual indicator to the remote location; and
the step of displaying the image of the retina at the remote location includes displaying the visual indicator on the retina at the remote location.

23. A system for correcting vision in the eye, comprising
an imaging device adapted to take images of a portion of the eye;
a laser adapted to be positioned relative to the eye and be fired at said portion of the eye;
a first transmitting device coupled to said imaging device and said laser, said first transmitting device adapted to transmit said images of said portion and the position of said laser to a remote location;
a first receiving device at said remote location and adapted to receive information transmitted by said first transmitting device;
a first monitor at said remote location and coupled to said first receiving device for displaying the images of said portion; and
a first control device at said remote location and adapted to control said position of said laser;
a second transmitting device at said remote location coupled to said control device and adapted to transmit control signals from said first control device; and
a second receiving device coupled to said laser and adapted to send said control signals to said laser to move said laser.

24. A system for correcting vision according to claim 23, wherein
said laser includes a visual marker, adapted to indicate the point at which a laser beam emitted from said laser would contact said portion.

25. A system for correcting vision according to claim 24, wherein
said first monitor is further adapted to display said visual marker indicating the point in which said laser beam emitted from said laser contacts said portion.

26. A system for correcting vision according to claim 24, further comprising
a second monitor coupled to said imaging device for displaying said images of said portion.

27. A system for correcting vision according to claim 26, wherein
said second monitor is further adapted to display said visual marker indicating the point in which said laser beam emitted from said laser contacts said portion.

28. A system for correcting vision according to claim 23, wherein
said portion of the eye is the fundus.

29. A system for correcting vision according to claim 28, wherein
said first control device is a joystick adapted to aim said laser at a desired portion of the fundus.

30. A system for correcting vision according to claim 23, further comprising
a second control device adapted to control said position of said laser.

31. A system for correcting vision according to claim 23, wherein
said transmitting device transmits information via satellite.

32. A system for correcting vision according to claim 23, wherein
said transmitting device transmits information via wired communication.

33. A system for correcting vision according to claim 23, wherein
said transmitting device transmits information via wireless communication.

34. A system for correcting vision according to claim 23, wherein
said laser is a coagulative laser.

35. A system for correcting vision according to claim 23, wherein
said laser is a disruptive laser.

36. A system for correcting vision according to claim 23, wherein
said laser is a thermal laser.
